(19) United States
(12) Patent Application Publication
(10) Pub. No.: US 2006/0251581 A1
(43) Pub. Date: Nov. 9, 2006

(54) Method for treatment of uterine fibroid tumors

(76) Inventors: Jon T. McIntyre, Newton, MA (US); Arthur Madenjian, Winchester, MA (US)

Correspondence Address: MAYER & WILLIAMS PC, 251 NORTH AVENUE WEST, 2ND FLOOR, WESTFIELD, NJ, 07090, US

(21) Appl. No.: 11/124,828

(22) Filed: May 9, 2005

(51) Int. Cl. A61K 49/00; A61K 49/04; A61K 9/14

(52) U.S. Cl. 424/9.3; 424/9.4; 424/9.5; 424/486

(57) ABSTRACT

Various injectable or insertable uterine fibroid treatment formulations are provided, which comprise a uterine fibroid treatment agent in an amount effective to cause shrinkage or elimination of uterine fibroids. The injectable or insertable formulations are typically solids, semi-solids or high-viscosity fluids. Other aspects of the invention are directed to systems and methods for treatment of uterine fibroids.

### STATEMENT OF RELATED APPLICATIONS

This application is related to U.S. patent application Ser. No. 10/667,151 entitled “Injectable Therapeutic Formulations,” filed Sep. 18, 2003, and U.S. patent application Ser. No. 10/664,601 entitled “Solid or Semi-Solid Therapeutic Formulations”, each filed Sep. 18, 2003, and each of which is hereby incorporated by reference.

### FIELD OF THE INVENTION

The present invention relates to formulations and methods for the treatment of unwanted tissue. More particularly, the present invention relates to formulations and methods for the shrinkage or elimination of uterine fibroid tumors.

### BACKGROUND OF THE INVENTION

Uterine fibroids tumors (also referred to as “uterine fibroids” or “leiomyomas”) are non-cancerous smooth muscle tumors of the uterus. They are believed to occur in 20 percent to 50 percent of women, or even more, depending on age and race. Uterine fibroids are a common cause of non-emergency uterine bleeding. Uterine fibroids also cause “bulk” symptoms such as low back pain and urinary frequency/urgency. They can also cause pain during intercourse (dyspareunia) and may cause problems with fertility and pregnancy.

Treatment of uterine fibroids costs billions of health care dollars each year and several treatments are available for this condition, depending on the type and severity of symptoms, as well as the number and location of the fibroids. Some of the common treatments are quite invasive. For example, although a hysterectomy results in the complete removal of the uterus; the majority of the hysterectomies in the United States each year are performed to treat uterine fibroids. Myomectomy is also commonly used to surgically remove uterine fibroids. However, about three-quarters of the myomectomy surgeries are open surgeries involving an abdominal incision.

A more recently developed treatment is uterine artery embolization. During this procedure, a catheter is inserted into a femoral artery and guided to a uterine fibroid artery. Small particles are then injected from the catheter into the fibroid artery, blocking its blood supply and causing it to eventually shrink and die. Although this procedure is less invasive than the above procedures, it commonly results in pain-related post-surgical symptoms.

Myolysis and cryomyolysis are techniques in which uterine fibroids are burned or frozen via laproscopic surgery. Like uterine artery embolization, myolysis/cryomyolysis causes fibroids to shrink and die over time. However, these procedures are rarely used because multiple punctures of the fibroids are required. In addition to charring at puncture sites, these punctures may give rise to post-surgical adhesions.

Hence, there is a continuing need for alternative therapies for the treatment of uterine fibroids, which are not open procedures and which preserve the patient's uterus.

### SUMMARY OF THE INVENTION

The above and other needs and challenges are addressed by the present invention. According to an aspect of the present invention, various injectable or insertable uterine fibroid treatment formulations are provided, which comprise a uterine fibroid treatment agent in an amount effective to cause shrinkage or elimination of uterine fibroids. The injectable or insertable formulations are typically solids, semi-solids or high-viscosity fluids.

Other aspects of the invention are directed to systems for treatment of uterine fibroids, which comprise: (a) an injectable or insertable uterine fibroid treatment formulation, such as those described herein, and (b) an apparatus for transcutaneously, transvaginally, or transcervically inserting the formulation into uterine fibroids. Still other aspects of the invention are directed to methods for treatment of uterine fibroids, which comprise: (a) providing an injectable or insertable uterine fibroid treatment formulation, such as those described herein, and (b) injecting or inserting the formulation into uterine fibroids.

One advantage of the present invention is that formulations, systems and methods are provided for the treatment of uterine fibroids, which do not result in open procedures and which preserve the patient's uterus.

Another advantage of the present invention is that injectable or insertable formulations are provided, which display improved retention of agents within uterine fibroid tissue, thereby improving delivery efficiency, while at the same time minimizing adverse effects such as nonspecific damage.

These and other embodiments and advantages of the present invention will become immediately apparent to those of ordinary skill in the art upon review of the Detailed Description and Claims to follow.

### BRIEF DESCRIPTION OF THE DRAWINGS

FIG. 1 is a schematic partial cross-sectional view of one embodiment of a device for delivery of a dosage form in accordance with the present invention.

### DETAILED DESCRIPTION OF THE INVENTION

According to an aspect of the present invention, injectable or insertable formulations are provided, which contain one or more uterine fibroid treatment agents in an amount effective to shrink or eliminate fibroids that are exposed to the formulation.

In some embodiments, the injectable or insertable formulations of the present invention are solids, semi-solids or high-viscosity fluids. This results, for example, in good dosage retention in the tissue (e.g., little to no back-leakage into the injection tract, control of injectable location to a specific region in the tissue, maintenance of a high concentration of treatment agent for a longer period of time, etc.), thereby improving delivery efficiency of the treatment agents and/or minimizing the adverse effects such as unintended, nonspecific tissue damage.

As the term suggests, “fluids” are materials that flow, meaning that these materials will deform (strain) continuously upon the application of a constant shear stress. As a result of their properties, fluids will take the shape of a container into which they are introduced. Moreover, they are capable of being injected into tissue (e.g., from a needle) upon application of sufficient stresses. Viscosity is a measure of a fluid's resistance to flow, with higher viscosity fluids having greater resistance to flow than lower viscosity fluids. “Highly viscous,” “high viscosity” and other such terms are used herein to describe fluids having viscosities greater than 1000 cps as measured by any of a number of techniques, including, for example, a Brookfield Kinematic Viscometer, model HBDV-II+CP with a CPE-40 cone spindle, set at 37° C. temperature, and using a 0.5 rpm speed setting. “Low viscosity” fluids have viscosities less than this value.

Unlike liquids, solids are materials that have a distinct unloaded shape (e.g., spherical, rod-shaped, cubic, etc.). Solid materials generally behave elastically when stress is applied (up to a point), with stress being proportional to strain. However, upon application of enough stress, solid materials eventually fail and break apart, for example, by brittle fracture or ductile fracture (in brittle fracture virtually no plastic deformation occurs before a crack propagates through the material, whereas in ductile fracture, considerable plastic deformation occurs before the material fractures). Examples of solid materials include pellets and polymers such as gelatin (below its melting point), each of which eventually fail with enough stress, although deformation can be quite significant in the case of the gelatin.

Other materials possess properties of both solids and fluids and are referred to herein as “semi-solids”. For example, a semi-solid material may behave as a solid (e.g., by having a distinct unloaded shape) until an applied stress exceeds a critical value (frequently referred to as the yield stress), after which the material begins to flow. Such materials are sometimes referred to as “viscoplastic fluids.” Examples include pastes and gels.

In some embodiments, a formulation in accordance with the present invention is provided ex vivo in a form that is readily retained by tissue (e.g., in a solid, semi-solid or high-viscosity fluid form), and it is subsequently injected or inserted into a patient.

In some embodiments, a formulation in accordance with the present invention is injected into a patient in a fluid state, whereupon it converts (or is converted) in vivo into a more readily retained form, for example, into a solid form (including conversion of an injected liquid into a solid, and conversion of an injected semi-solid into a solid), into a semi-solid form (including conversion of an injected liquid into a semi-solid, and conversion of an injected semi-solid into a semi-solid having increased yield stress and/or viscosity), or into a high-viscosity fluid (including conversion of a low-viscosity fluid into a high-viscosity fluid, and conversion of a high-viscosity fluid into a higher-viscosity fluid).

Uterine fibroid treatment agents for inclusion in the formulations of the present invention include chemical ablation agents, non-steroidal anti-inflammatory drugs (NSAIDs), oral contraceptives and GnRH agonists. The amount of uterine fibroid treatment agents used (e.g., the concentration and total dose) will vary widely, based on the nature of the uterine fibroid treatment agent that is selected and the nature of the formulation, among other factors, and is readily determinable by those of ordinary skill in the art.

Chemical ablation agents are materials whose inclusion in the formulations of the present invention in effective amounts results in necrosis (death) or shrinkage of nearby tissue upon injection or insertion of the formulation into the tissue. A wide range of ablation agent concentrations may be utilized in the formulations of the present invention, with the amounts employed being readily determined by those of ordinary skill in the art. Typical concentration ranges are from about 1 to 95 wt % of ablation agent, more typically about 5 to 80 wt %.

In some embodiments of the invention, the ablation agents are osmotic-stress-generating agents, for example, a salt, such as sodium chloride or potassium chloride. The process of osmosis is the passage of at least one diffusible species (commonly, water) through a semi-permeable membrane (e.g., the membranes that surround all cells in the body), which membrane simultaneously prevents the passage of at least one non-diffusible species (e.g., salt). In osmosis, the passage of the diffusible species is from a less concentrated solution (with respect to the non-diffusible species) through the membrane to a more concentrated one. What determines the relative concentration of the diffusible species is the amount of non-diffusible species present on either side of the membrane. Osmotic pressure is generated whenever environments of different water concentration are separated by a semi-permeable membrane, and will remain until the two solutions are of equal concentration. This is why cells frequently swell (and even burst, in some cases), when placed in distilled water, and why they frequently shrivel when placed in aqueous solutions containing high concentrations of a non-diffusible agent, such as salt (or when exposed to pure salt). If cells are subjected to sufficient osmotic stress, they will die.

In other embodiments, the ablation agents are organic compounds, for example, ethanol, which is toxic in high concentrations while being non-toxic at lower concentrations. It is noted that alcohols, such as ethanol, like salt, can also dehydrate cells and tissues causing them to shrink and die.

In other embodiments, the ablation agents are basic agents such as sodium hydroxide and potassium hydroxide, acidic agents such as acetic acid and formic acid, and/or enzymes such as collagenase, hyaluronidase, pronase, and papain.

In still other embodiments, the ablation agents are free-radical generating agents, for example, hydrogen peroxide, potassium peroxide, or other agents that can form free radicals in uterine fibroid tissue. Upon formation, the free radicals will attack the tissue to create necrosis. For example, free radicals can be formed by decomposition of the free-radical generating agent upon exposure to water, exposure to heat, exposure to light and/or exposure to other agents.

In still other embodiments, oxidizing agents, such as sodium hypochlorite, hydrogen peroxide or potassium peroxide, tissue fixing agents, such as formaldehyde, acetaldehyde or glutaraldehyde, or coagulants, such as gengpin, and combinations thereof, are used as ablation agents.

Examples of non-steroidal anti-inflammatory drugs for use as uterine fibroid treatment agents include: aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid and its derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as ε-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts and esters thereof; as well as combinations thereof.

Examples of contraceptives for use as uterine fibroid treatment agents include: desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, pharmaceutically acceptable salts and esters thereof, and combinations thereof. Specific examples include: norethynodrel/mestranol (e.g., ENOVID™), ethinyl estradiol/norethindrone (e.g., LOESTRIN™, BREVICON™, MODICON™, GENORA™, NELONA™, NORINYL™, OVACON-35™ and OVACON-50™), ethinyl estradioulevonorgestrel (e.g., LEVLEN™, NORDETTE™, TR1-LEVLEN™ and TRIPHASIL-21™), ethinyl estradiol/norgestrel (e.g., LO/OVRAL™ and OVRAL™), ethinyl estradiol/ethynodiol diacetate (DEMULEN™), norethindrone/mestranol (e.g., NORINYL™, ORTHO-NOVUMO, NORETHIN™, GENORA™, and NELOVA™), ethinyl estradiovdesogestrel (e.g., DESOGEN™ and ORTHO-CEP™), ethinyl estradiol/norgestimate (e.g., ORTHO-CYCLEN™ and ORTHO-TRICYCLEN™), norethindrone (e.g., MICRONOR™ and NOR-QD™), and norgestrel (e.g., OVRETTE™).

Examples of GnRH agonists for use as uterine fibroid treatment agents include: buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, further pharmaceutically acceptable salts and esters thereof, and combinations thereof.

Further uterine fibroid treatment agents in addition to the chemical ablation agents, NSAIDs, oral contraceptives, and GnRH agonists discussed above include antiprogestogens, such as mifepristone, and selective progesterone receptor modulators (SPRMs), such as asoprisnil, among other agents. Combinations of the foregoing are also used.

In some embodiments, formulations for use in connection with the present invention consist essentially of one or more uterine fibroid treatment agents. A pure (or nearly pure) salt pellet is one specific example of such a formulation.

In other embodiments, the formulations for use in connection with the present invention contain various optional agents in addition to one or more uterine fibroid treatment agents.

For example, one or more biodisintegrable binders may be included in the formulations of the present invention, typically in connection with dosage forms having solid characteristics. Where employed, a wide range of biodisintegrable binder concentrations may be utilized, with the amounts varying based, for example, on the desired physical characteristics of the resulting dosage form and on the characteristics of the uterine fibroid treatment agent that is selected (e.g., the degree of dilution, release delay, etc. that is desired/tolerated), among other considerations. The concentration of biodisintegrable binder that is used can vary widely. Typical ranges are from about 1 to 80 wt % of biodisintegrable binder, more typically about 5 to 50 wt %.

A “biodisintegrable” material is one that, once placed in tissue such as uterine tissue, undergoes dissolution, degradation, resorption and/or other disintegration processes. Where such materials are included, formulations in accordance with the present invention will typically undergo at least a 10% reduction in weight after residing in tissue such as uterine tissue for a period of 7 days, more typically a 50-100% reduction in weight after residing in the tissue for a period of 4 days.

Biodisintegrable binders for use in connection with the present invention include biodisintegrable organic compounds, such as glycerine, and biodisintegrable polymers. As the term is used herein, a polymer can consist of as few as two monomeric units, although polymers commonly contain many more than two monomeric units. The biodisintegrable polymers for use in conjunction with the present invention can be of natural or synthetic origin, can be homopolymers or copolymers, and can be selected, for example, from the following: cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, hydroxyethyl starch (HES), pentosan polysulfate (xylan polysulfate) and other modified carbohydrates, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(ε-caprolactone-co-glycolide), carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide and poly(ethylene oxide-propylene oxide) (e.g., Pluronic acid from BASF), polyoxyethylene (polyethylene glycol), polyanhydrides, polyvinylaichol, polyethyleneamine and polypyrridine, additional salts and copolymers beyond those specifically set forth above, and blends of the forgoing (including mixtures of polymers containing the same monomers, but having different molecular weights).

In other embodiments, the solid is in the form of a super-cooled material (i.e., the material is provided at a temperature that is below the freezing point of water). One example of a super-cooled material is dry ice (frozen carbon dioxide) whereby the material causes physical damage to the cells, resulting in tissue necrosis, upon direct insertion into the fibroid.

Other embodiments of the invention are directed to formulations having fluid characteristics, including liquid formulations and semi-solid formulations such as viscoplastic fluid formulations (as discussed above, such formulations behave as solids at stresses below their yield stresses, and as fluids at stresses beyond their yield stresses).

These formulations typically contain one or more liquid species (e.g., water, one or more organic solvents, or a combination of water and one or more organic solvents). In embodiments where an organic solvent is present, this species can also act as an ablation agent for the formulation. Ethanol is one specific example of one such species.

In addition to one or more liquid species these formulations can also optionally contain one or more viscosity adjusting agents. Where used, viscosity adjusting agent(s) are typically present in an amount effective to provided the formulation with the desired viscosity, for example, by rendering the formulation highly viscous, for example, in an amount effective to provide a viscosity between about 5,000 and 200,000 cps, more typically between about 10,000 and 100,000 cps, and even more typically between about 20,000 and 40,000 cps. By providing formulations having viscosities within these ranges, the formulations remain capable of being injected into tissue, such as uterine tissue, using conventional injection equipment (e.g., syringes). However, due to their elevated viscosities, the formulations have improved retention within the tissue at the injection site. The concentration of the viscosity adjusting agent(s) that is(are) used can vary widely. Commonly, the overall concentration of the viscosity adjusting agent(s) is between about 1 and 20 wt %.

In many embodiments, the viscosity adjusting agents are polymers, which may be of natural or synthetic origin and are typically biodisintegrable. The polymers are also typically water soluble and/or hydrophilic. However, in some embodiments, for instance where an organic solvent such as dimethylsulfoxide (DMSO) is utilized as a liquid component, the viscosity adjusting agent can be relatively hydrophobic. The polymeric viscosity adjusting agents include homopolymers, copolymers and polymer blends.

Examples of viscosity adjusting agents for the practice of the present invention include the following: cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, hydroxyethyl starch (HES), dextran, dextran derivatives, chitosan, and alginic acid and its various salts, carrageenan, various gums, including xanthan gum, guar gum, gum arabic, gum karaya, gum ghatti, konjac and gum tragacanth, glycosaminoglycans and proteoglycans such as hyaluronic acid and its salts, heparin, heparin sulfate, dermatan sulfate, proteins such as gelatin, collagen, albumin, and fibrin, other polymers, for example, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide and poly(ethylene oxide-propylene oxide) (e.g., Pluronic acid from BASF), polyoxyethylene (polyethylene glycol), polyethyleneamine and polypyrridine, poly-metaphosphate (Kurrol salts), polyvinyl alcohol, additional salts and copolymers beyond those specifically set forth above, and blends of the foregoing (including mixtures of polymers containing the same monomers, but having different molecular weights), and so forth. (As seen from the above discussion, many of these species are also useful as binders.)

In other embodiments of the invention, formulations are crosslinked, either ex vivo or in vivo. Crosslinking is advantageous, for example, in that it acts to improve formulation retention (e.g., by providing a more rigid/viscous material and/or by rendering the polymer less soluble in a particular environment).

Where the formulation is crosslinked in vivo, a crosslinking agent is commonly injected into tissue either before or after the injection or insertion of a formulation in accordance with the present invention. Depending on the nature of the formulation and the crosslinking agent, the formulation may be converted, for example, into a solid, into a semi-solid, or into a high-viscosity fluid.

Crosslinking agents for use in the present invention include ionic and covalent crosslinking agents. For example, in some embodiments, polymers are included within the formulations of the present invention, which are ionically crosslinked, for instance, with polyvalent metal ions. Suitable crosslinking ions include polyvalent cations selected from the group consisting of calcium, magnesium, barium, strontium, boron, beryllium, aluminum, iron, copper, cobalt, lead and silver cations ions. Polyvalent anions include phosphate, citrate, borate, succinate, maleate, adipate and oxalate anions. More broadly, crosslinking anions are commonly derived from polybasic organic or inorganic acids. Ionic crosslinking may be carried out by methods known in the art, for example, by contacting ionically crosslinkable polymers with an aqueous solution containing dissolved ions.

In some embodiments, polymers are included, which are covalently crosslinkable, for example, using a polyfunctional crosslinking agent that is reactive with functional groups in the polymer structure. The polyfunctional crosslinking agent can be any compound having at least two functional groups that react with functional groups in the polymer. Various polymers described herein can be both covalently and ionically crosslinked.

Suitable polymers for ionic and/or covalent crosslinking can be selected, for example, from the following: polyacrylates; poly(acrylic acid); poly(methacrylic acid); polyacrylamides; poly(N-alkylacrylamides); polyalkylene oxides; poly(ethylene oxide); poly(propylene oxide); poly(vinyl alcohol); poly(vinyl aromatics); poly(vinylpyrrolidone); poly(ethylene imine); poly(ethylene amine); polyacrylonitrile; poly(vinyl sulfonic acid); polyamides; poly(L-lysine); hydrophilic polyurethanes; maleic anhydride polymers; proteins; collagen; cellulosic polymers; methyl cellulose; carboxymethyl cellulose; dextran; carboxymethyl dextran; modified dextran; alginates; alginic acid; pectinic acid; hyaluronic acid; chitin; pullulan; gelatin; gellan; xanthan; carboxymethyl starch; hyxdroxyethyl starch; chondroitin sulfate; guar; starch; and salts, copolymers, mixtures and derivatives thereof.

In other embodiments of the invention, formulations are used, which exist (a) as a liquid at temperatures below body temperature and (b) as a gel at body temperature. The temperature at which a transition from liquid to gel occurs is sometimes referred to as the lower critical solution temperature (LCST), and it can be a small temperature range as opposed to a specific temperature. Materials possessing an LCST are referred to as LCST materials. Typical LCST's for the practice of the present invention range, for example, from 10 to 37° C. As a result, a formulation injected below the LCST warms within the body to a temperature that is at or above the LCST, thereby undergoing a transition from a liquid to a gel.

Suitable LCST materials include polyoxyethylene-polyoxypropylene (PEO-PPO) block copolymers. Two acceptable compounds are Pluronic acid F127 and F108, which are PEO-PPO block copolymers with molecular weights of 12,600 and 14,600, respectively. Each of these compounds is available from BASF of Mount Olive, N.J. Pluronic acid F108 at 20-28% concentration concentration, in phosphate buffered saline (PBS) is an example of a suitable LCST material. One beneficial preparation is 22.5% Pluronic acid F108 in PBS. A preparation of 22% Pluronic acid F108 in PBS has an LCST of 37° C. Pluronic acid F127 at 20-35% concentration in PBS is another example of a suitable LCST material. A preparation of 20% Pluronic acid F127 in PBS has an LCST of 37° C. Typical molecular weights are between 5,000 and 25,000, and, for the two specific compounds identified above are 12,600 and 14,600. More generally, materials, including other PEO-PPO block copolymers, which are biodisintegrable, and which exist as a gel at body temperature and as a liquid below body temperature can also be used according to the present invention. Further information regarding LCST materials can be found in U.S. Pat. Nos. 6,565,530 B2 and 6,544,227 B2, each to Sahatjian et al., and each of which is hereby incorporated by reference.

In still other embodiments, formulations are used which have fluid characteristics at temperatures above a predetermined temperature, while having solid characteristics at temperatures below that temperature. An example is a biodisintegrable polymer formulation which has a melting point somewhat above body temperature, typically between 37 and 55° C. The liquid/solid transition can occur over small range of temperatures, as opposed to a single temperature. A formulation with such characteristics can be injected into the body at elevated temperature, after which it is cooled to body temperature (or below). During the cooling process, the formulation undergoes a phase transition to a solid form. In some embodiments, the formulation is actively cooled after it is injected into the patient's body.

One specific example of such a formulation is a gelatin-containing formulation, which is injected into the body as a fluid and which upon cooling becomes a solid. If desired, cooling of the gelatin material can be performed, for example, by concurrently injecting (a) a liquid gelatin-containing formulation at a temperature above body temperature and (b) another liquid (e.g., water or a buffer) at a temperature below body temperature.

In addition to a uterine fibroid treatment agent and any of the various optional components discussed above, the uterine fibroid formulations of the present invention also optionally include one or more imaging contrast agents.

The ability to non-invasively image regions where the formulations of the present invention are being introduced (and where they have been introduced) is a valuable diagnostic tool for the practice of the present invention. Among such currently available non-invasive imaging techniques are included magnetic resonance imaging (MRI), ultrasonic imaging, x-ray fluoroscopy, nuclear medicine, and others. Various categories of imaging technology have associated with them imaging contrast agents, i.e., substances that enhance the image produced by medical diagnostic equipment.

For example, x-ray based fluoroscopy is a diagnostic imaging technique that allows real-time patient monitoring of motion within a patient. To be fluoroscopically visible, formulations are typically rendered more absorptive of x-rays than the surrounding tissue. In various embodiments of the invention, this is accomplished by the use of contrast agents. Examples of contrast agents for use in connection with x-ray fluoroscopy include metals, metal salts and oxides (particularly bismuth salts and oxides), and iodinated compounds. More specific examples of such contrast agents include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine.

Ultrasound and magnetic resonance imaging can provide two-and/or three-dimensional images of a portion of the body. Ultrasound and MRI are advantageous, inter alia, because they do not expose the patient or medical practitioner to harmful radiation and they can provide detailed images of the observed area. These detailed images are valuable diagnostic aids to medical practitioners and can be used to more precisely control the quantity and location of the formulations of the present invention.

Ultrasound uses high frequency sound waves to create an image of living tissue. A sound signal is sent out, and the reflected ultrasonic energy, or “echoes,” used to create the image. Ultrasound imaging contrast agents are materials that enhance the image produced by ultrasound equipment. Ultrasonic imaging contrast agents introduced into the formulations of the present invention can be, for example, echogenic (i.e., materials that result in an increase in the reflected ultrasonic energy upon injection or insertion of the formulation) or echolucent (i.e., materials that result in a decrease in the reflected ultrasonic energy upon injection or insertion of the formulation).

Suitable ultrasonic imaging contrast agents for use in connection with the present invention include solid particles ranging from about 0.01 to 50 microns in largest dimension (e.g., the diameter, where spherical particles are utilized), more typically about 0.5 to 20 microns. Both inorganic and organic particles can be used. Examples include microparticles/microspheres of calcium carbonate, hydroxyapatite, silica, poly(lactic acid), and poly(glycolic acid). Microbubbles can also be used as ultrasonic imaging contrast agents, as is known in the imaging art. The ultrasonic imaging contrast agents for use in connection with the present invention are preferably biocompatible and stable in the formulation. Concentrations of the ultrasonic imaging contrast agents typically range from about 0.01 wt % to 10 wt % of the formulation, more typically about 0.05 to 2 wt %, where solid particles are used.

Magnetic resonance imaging (MRI) produces images by differentiating detectable magnetic species in the portion of the body being imaged. In the case of ¹H MRI, the detectable species are protons (hydrogen nuclei). In order to enhance the differentiation of detectable species in the area of interest from those in the surrounding environment, imaging contrast agents are often employed. These agents alter the magnetic environment of the detectable protons in the area of interest relative to that of protons in the surrounding environment and, thereby, allow for enhanced contrast and better images of the area of interest. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gadolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have been employed as MRI contrast agents. Chelation of the gadolinium or other paramagnetic ion is believed to reduce the toxicity of the paramagnetic metal by rendering it more biocompatible, and can assist in localizing the distribution of the contrast agent to the area of interest. Paramagnetic ion chelates can be, for example, attached to formulation components (e.g., attached to binders, viscosity adjusting agents, etc.) or they can simply be admixed with the other components of the formulation. Further information can be found, for example, in U.S. Patent Application No. 20030100830 entitled “Implantable or insertable medical devices visible under magnetic resonance imaging,” the disclosure of which is incorporated herein by reference.

Formulations in accordance with the present invention may be formulated by a variety of methods.

For example, formulations of the present invention having liquid attributes can be provided by simply admixing: (a) water, one or more organic solvents, or a combination of water and one or more organic solvents, (b) a fibroid treatment agent if necessary (as noted above, some organic solvents can also act as fibroid treatment agents), and (c) any other ingredients, as desired, such as optional viscosity adjusting agents, imaging contrast agents, and so forth.

As another example, formulations of the present invention having solid attributes can be provided using fluid processing techniques such as melt processing techniques and solution/suspension processing techniques. For instance, in some embodiments, the formulations will have thermoplastic characteristics (e.g., by virtue of the presence of one or more thermoplastic components, such as thermoplastic polymers), thereby allowing melts to be formed. In other embodiments, solutions or dispersions (including slurries and pastes) of various formulations components are formed. The resulting fluid (i.e., the melt, solution or dispersion) can then be further processed, as desired.

For example, in some instances, the resulting fluid is poured into a mold and solidified (e.g., by cooling the melt, or by evaporating solvent from the solution/dispersion). The resulting solidified mass can then be used in its molded shape or it can be processed further (e.g., by grinding, breaking, cutting, sculpting, etc.) to achieve a desired character.

In other instances, the resulting fluid can be extruded, dripped, sprayed, etc., at which point it is solidified, for example, by cooling the melt (e.g., by contact with a cooled gas or liquid), by removing solvent from the solution/dispersion (e.g., by contact with a heated gas or contact with another solvent), or by crosslinking the solution/dispersion (e.g., by contact with a solution containing a crosslinking agent), among other techniques. As above, the resulting mass can then be used as is, or it can be further processed to achieve a desired character. Where extrusion is employed, it is possible to co-extrude one or more coating layers on an underlying extrudate.

In yet other instances, the melt or solution/dispersion is mixed with a larger volume of an immiscible solvent, whereupon sufficient shear is applied to disperse the melt or solution/dispersion within a continuous phase occupied by the immiscible solvent. The dispersed phase (occupied by the melt or solution/dispersion) is subsequently stabilized, for example, (a) by cooling the dispersed melt to form a solid dispersion, (b) by coating or crosslinking the outer surface of the dispersed solution/dispersion, or (c) by removing the solvent from the dispersed solution/dispersion. As above, the resulting beads or particles can then be used as produced, or they can be processed further to achieve a desired character.

Where the formulations of the present invention have solid characteristic, they can be provided in an essentially unlimited variety of shapes (e.g., spheres, cylinders, and irregular shapes including beads of various shapes) and sizes (e.g., having largest dimensions ranging from 1 micron to 3 microns to 10 microns to 30 microns to 100 microns to 300 microns to 1 mm to 3 mm to 30 mm and all ranges in between). For example, where pellets are utilized, they typically have a largest dimensions ranging from 1 to 20 mm. As a more specific example, cylindrical pellets can be provided, which have an outside diameter ranging from 1 to 3 mm and a length ranging from 1 to 20 mm.

Where the formulations of the present invention have solid characteristic, they can also be coated, for example, to protect the formulations, or to delay delivery of the uterine fibroid treatment agent (e.g., until the formulations are properly positioned within the patient). The formulations can be fully coated or partially coated. For instance, it may be desirable to coat only the forward (relative to the direction of insertion) or rearward portions of the formulations. Beneficial materials for coating the formulations include biodisintegrable polymers such as those discussed above. Formulations in accordance with the present invention can be coated in some embodiments by dipping in or spraying with a polymer dispersion or solution, followed by drying or crosslinking.

Prior to injection or insertion, the formulations of the present invention are typically rendered sterile, for example, by exposing them to heat, radiation or ethylene oxide gas, or by preparation under aseptic conditions.

The formulations of the present invention are injected/inserted into uterine tissue in a variety of forms, by a variety of routes, using a variety of apparatuses. Subjects (also referred to herein as “patients”) for the procedures of the present invention include vertebrate subjects, typically mammalian subjects, and more typically human subjects.

In some embodiments, the formulations of the present invention are injected into uterine fibroid tumors using a hollow delivery channel, such as a hollow needle or cannula. For instance, in some embodiments, a needle is used in association with a conventional or specially designed syringe, cannula, catheter, and so forth.

In some embodiments, a source of pressure (e.g., a conventional syringe plunger, a pump, aerosol, etc.) is utilized to inject the formulation into the fibroid.

Where the formulation has solid attributes, it is injected/inserted in certain embodiments by forcing the formulation through a hollow delivery channel into the fibroid, for example, using mechanical, hydraulic, pneumatic, or other action.

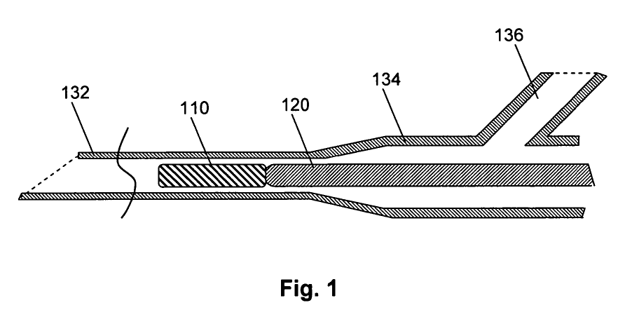

As a specific example, FIG. 1 is a cross-sectional illustration of an apparatus, which comprises a body 134 and a needle 132. The body is further provided with a side port 136 through which a formulation having solid characteristics is introduced. Subsequently, a sampler pusher 120 (e.g., a mandrel or modified obturator) is used to push the formulation 110 into the barrel of the needle 132. If desired, an additional side port (not illustrated) can be employed for introduction of the sample pusher 120. Once the needle 132 is inserted to the desired position within the patient's tissue (not illustrated), the sampler pusher 120 is used to push the formulation 110 from the needle 132 into the tissue, after which the needle 132 is withdrawn. In some instances it may be desirable to withdraw the needle by a short distance, allowing the formulation to be pushed into an opening in the tissue created by the needle. In some embodiments, stoppers (not illustrated) are employed to control the depth to which the needle is inserted and/or the depth to which the sample pusher is inserted.

In other even more straightforward embodiments the formulation is injected/inserted using an apparatus consisting of a simple needle (e.g., a 10 gauge or smaller needle) and sample pusher (e.g., a mandrel or modified obturator). For example, according to one embodiment, a formulation (e.g., a rod-shaped formulation or beads) is placed in the needle. Once the needle is placed at the desired depth and location in the tissue, the pusher is used to push the sample from the needle and into the tissue. In some embodiments, the sample pusher is provided with a holding clip or it is provided with a hollow end to secure the sample up to the time of delivery.

In other embodiments, a device is employed that cores out a section of the fibroid (e.g., a biopsy device or tissue morcellator or laser radiation), thereby leaving behind a void for insertion of a dosage form.

In still other embodiments, formulations in accordance with the present invention are injected/inserted via jet injection. Jet injection provides a method of administering the formulations without the use of delivery channels such as needles. Typically, a compression system (e.g., a mechanical system or a gas, such as helium, nitrogen, carbon dioxide, etc.) is used to accelerate the formulations to a relatively high velocity, allowing them to penetrate the tissue. Jet injector devices can be, for example, disposable, or reusable with medication cartridges that are prefilled or non-prefilled medication cartridges. Examples of jet injectors include Biojector® from Bioject, N.J., USA and the PowderJect® System from PowderJect, UK. In addition, such injection methods enable dispersion control (depth and width) of the injectate.

Injection routes include transabdominal and transvaginal routes. Where the formulations have fluid attributes, the injection volume will vary, depending, for example, on the size of the fibroid, the type and concentration of treatment agent, and so forth, and will typically range from 1.0 to 10.0 ml per injection. Similarly, where formulations having solid attributes (e.g., pellets or powders) are utilized, the amount of formulation injected/inserted will also depend, for example, on the size of the fibroid, the type and concentration treatment agent utilized, etc. Multiple pellets can be inserted at a single injection site. Regardless of the physical attributes of the formulation, multiple injection/insertion sites may be established within a single fibroid, with the number of injections depending on the size and shape of the fibroid as well as the type and/or concentration of the treatment agent that is used. Multiple fibroids can also be treated.

To further enhance the delivery capabilities of the present invention, an energy source is applied in some embodiments to the fibroid after delivery of the formulation, for example, to initiate, accelerate or terminate delivery of the fibroid treatment agent. Examples of energy sources include ultrasound sources, microwave sources, radio-frequency sources, and light sources.

In various embodiments, the injection/insertion device is guided to the fibroid site under image guidance. Image guidance can include, for example, direct visual guidance (e.g., laparoscopic guidance in trans-abdominal procedures and hysteroscopic guidance in trans-vaginal procedures) and non-direct visual guidance (e.g., ultrasound guidance, fluoroscopic guidance, and/or MRI guidance). The ability to visually image the injection/insertion device and the inserted/injected formulation by non-direct techniques will clearly depend upon the contrast of the device and formulation with the surrounding tissue. For example, metallic injection/insertion devices are commonly readily visible under ultrasonic, fluoroscopic and magnetic resonance imaging, and various contrast agents can be added to the formulations of the present invention to enhance visibility as discussed above.

As a specific example, visual guidance of the injection/insertion device is conducted laparoscopically using a scope that is positioned in the abdomen (e.g., by insertion through a trocar). In this way, a device (e.g., a delivery needle) can be inserted percutaneously into the abdomen and guided under laproscopic vision to the uterine fibroid. Once the fibroid is reached, fluoroscopy, MRI or ultrasound (e.g., trans-vaginal ultrasound, trans-abdominal ultrasound, intra-abdominal ultrasound, etc.) is used to guide the tip of the delivery needle to a desired position within the fibroid, at which point the formulation is injected or inserted into the fibroid. To the extent that there is sufficient contrast between the formulation and the surrounding tissue, the location of the formulation within the fibroid will also be viewed.

The invention is further described with reference to the following non-limiting Examples.

### EXAMPLES

### Example 1

5 g of carboxymethylcellulose 7HF PH (CMC, Blanose Type, Hercules Inc.) is dissolved in 100 ml of saturated sodium chloride solution (˜26% w/w). The polymer is then extruded through an 18G needle into a 70% ethanol solution to form a rod/filament. The sodium chloride solution may be over-saturated, with the excessive sodium chloride particles suspended in the thick paste of CMC. The dried rod/filament is cut to desired length, typically 1 cm, for easy delivery.

### Example 2

The formulations produced in Example 1 are spray-coated with one or more layers of an aqueous solution of CMC (˜1% w/w) to increase the strength of the formulations. The coated formulations are then air dried, or they are dehydrated in ethanol followed by air drying.

### Example 3

50 ml of CMC/sodium chloride solution from Example 1 and 50 ml of a 3 wt % sodium alginate (from Protanal, LF 10/60) aqueous solution are mixed thoroughly. The mixture is extruded into absolute ethanol bath to provide a solid rod. The rod is then crosslinked using a calcium chloride solution (e.g., at a concentration of 30 wt %) by dipping the rod into the CaCl₂ bath. Excess calcium chloride is removed by rinsing in ethanol.

### Example 4

The mixture from Example 3 is extruded into a 30 wt % calcium chloride bath, instead of absolute ethanol, to form a solid rod/filament. This filament/rod is then air dried and cut into 1 cm lengths for delivery.

### Example 5

The mixture from Example 3 is dispersed in a mineral oil or a silicone oil bath under agitation. Microbeads are formed in the bath. The size of the beads may be controlled by the speed of the agitation. 30 wt % calcium chloride solution is added into the bath to crosslink the alginate beads. The reaction is allowed to continue overnight to obtain solid beads. The beads are collected and washed in ethanol. After thorough drying, the beads are ready for delivery.

### Example 6

Sodium alginate is dissolved in water forming a first solution. CMC is dissolved in salt solution as above to form a second solution. The two solutions are co-extruded, with the inner extrudate (which emerges from the extrusion die in the shape of a rod) containing CMC and salt, and the outer extrudate (which emerges from the extrusion die in the shape of a tube) containing alginate and forming a crosslinkable coating around the inner extrudate. The coextruded product is dropped into a calcium chloride bath to crosslink the alginate in the coating. The crosslinked material is then dried. Washing the material in ethanol can accelerate the drying.

### Example 7

2 g of CMC powder and 8 g of sodium chloride powder are mixed thoroughly. About 8 g of water are added dropwise into the mixed powder with stirring. A uniform thick paste is formed. The paste is then extruded through the opening of a 5 ml syringe (Becton Dickinson Co.) to form a uniform filament/rod. The filament/rod is allowed to dry in the air at room temperature overnight. The dried filament/rod has an OD of about 1 mm. It is cut into 1 cm lengths in preparation for use.

### Example 8

The same mixing, extrusion and drying procedures as set forth in Example 7 are used with the following: 2 g dextran sulfate (from Bioworld), 8 g Sodium Chloride and 2 g water. A solid filament/rod is obtained and cut. The resulting rod has the following dimensions: OD=1 mm, length 1 cm.

### Example 9

A solution is formed that contains 10 wt % of poly(DL-lactide-co-glycolide (from Aldrich, lactide:glycolide, molar ratio=85:15) dissolved in THF. The rods from Examples 7 and 8 are coated with this solution. The rods are dipped into the solution three times to achieve the desired coating thickness. Alternatively, a spray coating technique may be used. The coating should be about 5 microns in thickness, and is allowed to dry in air at room or elevated (e.g., 50° C.) temperature. The dried coated rods are cut into 1 cm lengths for use (if not cut prior to coating).

### Example 10

An absorbable polymer, for example, PGA (poly-glycolic acid) is compounded with NaCl to form a matrix. This may be carried out, for example, using a Baybender compounding machine (or other compounder) to mix and blend the components. The resulting compounded mixture is then extruded or molded into various forms for implantation. The breakdown of the PGA within the body will expose and release the salt. Compounding is beneficial in that it potentially provides a greater polymer surface area for breakdown of the structure and absorption of the polymer. An added potential benefit of compounding the salt into the polymer is the delayed release of the salt (i.e., it can be dependent upon to polymer degradation). On the other hand, if the concentration of salt is high enough to create contiguous pockets within the matrix structure (i.e., producing a sponge-like structure), rapid breakdown of the polymer is expected to occur after the salt is gone.

### Example 11

Polyvinylpyrolidone (PVP) (K 90, BASF, Product # 09608802) is added to absolute, anhydrous ethanol (anhydrous 99.57%, Aldrich) while mixing in a beaker, wide mouth bottle or plastic jar using overhead stirrer with variable speed settings. The formulation is completed by stirring in calcium carbonate (CaCO₃)(EM Industries, Germany, catalog # EMCX0127-1). Formulation ranges are as follows:

PVP from 5% to 25 wt %

Ethanol 40% to 100 wt %

CaCO₃ 0.05% to 10 wt %

Viscosity is measured using a Brookfield Kinematic Viscometer with CPE-40 cone spindle set at 0.5 rpm and 37° C. temperature, and found to be between 500 and 20000 cps.

### Example 12

5 wt % sodium alginate (FMC Biopolymer, Protonal LF 10/60) is dissolved in 30 grams D.I. water. Subsequently 7.5 grams of Sodium Chloride (NaCl) (VWR Scientific) are added, while mixing as described in Example 11, to form a gel. The formulation is completed by mixing in 1 wt % calcium carbonate (CaCO₃).

### Example 13

33,000 mg of salt is dissolved in 100 ml of DI water by mixing in a wide mouth glass or plastic jar. Subsequently, 3100 mg of CMC (Hercules Inc., Blanose Type 7HF PH, 9M31F PH or 7MF) polymer is quickly added into the salt solution to form a gel. The formulation is completed by mixing in 1 wt % calcium carbonate (CaCO₃). This particular formulation contains 1.30 wt % CMC, 13.84 wt % NaCl and 1 wt % calcium carbonate. General formulation ranges are as follows:

CMC from 1 wt % to 4 wt %

NaCl from 5 wt % to 30 wt %

CaCO₃ from 0.05 wt % to 10 wt %

Viscosities for these formulations range from 29,000 to 36,000 cps.

### Example 14

About 3% by weight of hydroxypropyl cellulose (HPC) (Hercules Inc., Klucel Type HF or Type MF, Pharmaceutical Grade) is slowly added to absolute, anhydrous ethanol while stirring in a wide-mouth glass or plastic jar. The formulation is completed by mixing in 1 wt % calcium carbonate (CaCO₃). In general, formulation ranges are as follows:

HPC from 1% to 10 wt %

Ethanol 40% to 100 wt %

CaCO₃ 0.05% to 10 wt % Viscosity is typically between 36,000 and 42,000 centipoises for a formulation having about 3 wt % Klucel Type HF HPC. More generally, viscosity typically ranges from about 12,000 to 47,000 cps for HPC concentrations ranging from about 2 to 5 wt %.

### Example 15

NaCl is added to D.I. water, followed by CMC in sufficient quantities to yield a NaCl-CMC solution containing 330 mg/ml NaCl (or to saturation) and 40 mg/ml CMC. At the same time an alginate solution is prepared by adding sodium alginate to water at a concentration of 75 mg/ml. Equal volumes of the NaCl-CMC solution and the alginate solution are then mixed in a wide-mouth glass or plastic jar to form a viscous gel. The resulting gel contains 2% w/v CMC, 24% w/v NaCl, and 2.5% w/v alginate (which corresponds, if dry, to 7% w/w CMC, 84.21% w/w NaCl, and 8.77% w/w alginate), for a target viscosity between 32,000 and 36,000 cps.

More water can be added to decrease viscosity as desired. Additional formulations can be prepared by substituting additional uterine fibroid treatment agents for the salt. Note that alginate is not soluble in high salt solution, hence the two step mixture. For other agents besides salt, the mixture should be more straightforward (e.g., adding all the ingredients into the water).

The formulation is then injected into a uterine fibroid. Subsequently, the needle is retracted and a crosslinker (e.g., 2-20% w/w CaCl₂ in distilled water) is injected to crosslink part of the injected material, increasing the resistance of the injected gel to back-leakage.

### Example 16

A sodium hydroxide (NaOH) solution ix mixed with hydroxyethyl starch (HES, Sigma, #H6382) to form a paste. The paste is injected into a mold and allowed to dry. The molded parts form a rod with the following dimensions: OD=1.5 mm, length=10 mm, and they contain 10 wt % NaOH and 90 wt % HES.

Although various embodiments are specifically illustrated and described herein, it will be appreciated that modifications and variations of the present invention are covered by the above teachings and are within the purview of the appended claims without departing from the spirit and intended scope of the invention.

1. A method for the treatment of uterine fibroids, said method comprising:
providing an injectable or insertable formulation that comprises a uterine fibroid treatment agent in an amount effective to cause shrinkage of uterine fibroids; and
injecting or inserting said formulation into the uterine fibroid.

2. The method of claim 1, wherein said formulation is injected or inserted into said fibroid transabdominally.

3. The method of claim 1, wherein said formulation is injected or inserted into said fibroid transvaginally

4. The method of claim 1, wherein said formulation is injected or inserted into said fibroid under image guidance.

5. The method of claim 4, wherein said image is at least one of a direct visual image and a non-direct visual image.

6. The method of claim 5, wherein said direct image is scope image.

7. The method of claim 5, wherein said non-direct visual image is an MRI image.

8. The method of claim 7, wherein said formulation comprises an MRI contrast agent.

9. The method of claim 5, wherein said non-direct visual image is an ultrasound image.

10. The method of claim 9, wherein said formulation comprises an ultrasound contrast agent.

11. The method of claim 5, wherein said non-direct visual image is a fluoroscopic image.

12. The method of claim 11, wherein said formulation comprises an x-ray contrast agent.

13. The method of claim 1, wherein said uterine fibroid treatment agent is selected from a chemical ablation agent, a non-steroidal anti-inflammatory drug, an oral contraceptive, a GnRH agonist, an antiprogestogen, and a selective progesterone receptor modulator.

14. The method of claim 1, wherein said uterine fibroid treatment agent is a chemical ablation agent.

15. The method of claim 14, wherein said chemical ablation agent is a salt.

16. The method of claim 14, wherein said chemical ablation agent is selected from an enzyme, an acid, a base and an oxidizing agent.

17. The method of claim 1, wherein said formulation comprises a plurality of different uterine fibroid treatment agents.

18. The method of claim 1, wherein said formulation is a solid formulation.

19. The method of claim 18, wherein said formulation comprises a biodisintegrable binder.

20. The method of claim 19, wherein said biodisintegrable binder is selected from a glycolic acid polymer, a cellulose ether, and a crosslinked polymer.

21. The method of claim 18, wherein said formulation is a dosage form having a largest dimension between 1 mm and 20 mm.

22. The method of claim 18, wherein said formulation is delivered through a hollow channel into said fibroid.

23. The method of claim 18, wherein said formulation is encapsulated.

24. The method of claim 1, wherein said formulation is a powder.

25. The method of claim 24, wherein said powder is introduced into said fibroid by jet injection.

26. The method of claim 1, wherein said formulation is a high-viscosity formulation.

27. The method of claim 26, wherein said formulation comprises a viscosity adjusting agent.

28. The method of claim 27, wherein said viscosity adjusting agent is present in an amount effective to provide a viscosity ranging from 10,000 cps to 50,000 cps.

29. The method of claim 1, wherein said formulation is a gel.

30. The method of claim 1, wherein said formulation is a fluid formulation that is converted in vivo into a solid, a semi-solid or a high-viscosity liquid.

31. The method of claim 1, wherein said formulation is a fluid formulation that is crosslinked in vivo.

32. The method of claim 31, wherein a crosslinking agent is injected into said fibroid in a step separate from the injection of said fluid formulation.

33. The method of claim 31, wherein said formulation is ionically crosslinked in vivo.

34. The method of claim 33, wherein said formulation comprises an alginate polymer.

35. The method of claim 1, wherein said formulation has a lower critical solution temperature (LCST) that is less than or equal to the body temperature of said patient, and wherein said formulation is injected at a temperature that is less than said lower critical solution temperature (LCST).

36. The method of claim 35, wherein said formulation comprises a polyoxyethylene-polyoxypropylene block copolymer.

37. The method of claim 1, wherein said formulation has a melting point that is greater than the body temperature of said patient, and wherein said formulation is injected at a temperature that is greater than said melting point.

38. The method of claim 37, wherein said formulation comprises gelatin.

39. A system for the treatment of uterine fibroids, said system comprising:
(a) an injectable or insertable formulation that comprises a uterine fibroid treatment agent in an amount effective to cause shrinkage of uterine fibroids; and
(b) an apparatus for injecting or inserting said formulation into said fibroids.

40. A fibroid injectable or insertable formulation that comprises a uterine fibroid treatment agent, selected from a non-steroidal anti-inflammatory drug, an oral contraceptive, a GnRH agonist, an antiprogestogen, and a selective progesterone receptor modulator, in an amount effective to cause shrinkage of uterine fibroids.
